(19) United States
(12) Patent Application Publication
(10) Pub. No.: US 2023/0260534 A1
(43) Pub. Date: Aug. 17, 2023

(54) SMART GLASS INTERFACE FOR IMPAIRED USERS OR USERS WITH DISABILITIES

(71) Applicant: Meta Platforms Technologies, LLC, Menlo Park, CA (US)

(72) Inventors: Johana Gabriela Coyoc Escudero, Milpitas, CA (US); Scott Phillip Selfon, Kirkland, WA (US); Simon Porter, San Jose, CA (US); Christi Miller, Seattle, WA (US); Yao Ding, San Mateo, CA (US); Jonathan Y. Lee, Brooklyn, NY (US); Gregory Sarkis-Kelly, San Diego, CA (US); Brett Alden Lavalla, Eugene, OR (US); FNU Anurag Kumar, Bellevue, WA (US)

(21) Appl. No.: 18/163,436

(22) Filed: Feb. 2, 2023

Related U.S. Application Data
(60) Provisional application No. 63/306,854, filed on Feb. 4, 2022.
(60) Provisional application No. 63/323,901, filed on Mar. 25, 2022.
(60) Provisional application No. 63/348,392, filed on Jun. 2, 2022.

(51) Int. Cl. G10L 25/51; H04R 1/02; H04R 5/027; H04R 3/00; H04S 1/00; H04R 3/04; G06F 40/58

(52) U.S. Cl. CPC G10L 25/51; H04R 1/028; H04R 5/027; H04R 3/005; H04S 1/007; H04R 3/04; G06F 40/58; H04S 2400/15; H04R 2201/401; H04N 23/57

(57) ABSTRACT

A headset designed for inclusion of users with impairments is provided. The headset includes a frame, two eyepieces mounted on the frame, and at least one microphone and a speaker, mounted on the frame. The headset also includes a camera, a memory configured to store multiple instructions, and a processor configured to execute the instructions, wherein the instructions comprise to provide to a user an environmental context from a signal provided by the microphone and the camera. A method for using the above headset and a system for performing the method are also provided.

### CROSS-REFERENCE TO RELATED APPLICATIONS

The present disclosure is related and claims priority under 35 U.S.C. § 119(e), to U.S. Prov. Appln. No. 63/306,854, entitled INTERFACE IN SMART GLASSES AND VR/AR DEVICES FOR IMPAIRED USERS OR USERS WITH DISABILITIES, filed on Feb. 4, 2022, to U.S. Prov. Appln. No. 63/323,901, entitled INTERFACE IN SMART GLASSES AND VR/AR DEVICES FOR IMPAIRED USERS OR USERS WITH DISABILITIES, FILED ON Mar. 25, 2022, and to U.S. Prov. Appln. No. 63/348,392, entitled SIGN LANGUAGE DETECTION FOR SMART GLASSES, filed on Jun. 2, 2022, all to Johana Gabriela Coyoc ESCUDERO, et al., the contents of which applications are hereinafter incorporated by reference in their entirety, for all purposes.

### BACKGROUND

### Field

The present disclosure is directed to smart glasses to allow verbally impaired or users with disabilities. More specifically, embodiments as disclosed herein are directed to smart glasses including a user interface that provides context and situational awareness to impaired users and users with disabilities.

### Related Art

In the field of wearable devices, little attention is paid to users with disabilities in the assumption that they encompass a small portion of the market. However, the addition of technical features aiding users with special needs may open new applications that the general public may benefit from. In the case of verbally impaired users, sign language detection offers a challenging proposition, as complex, three-dimensional pattern recognition is desirable with high resolution (e.g., a few millimeters error in image recognition could render the effort moot) and at a relatively high pace (at least at an acceptable conversational speed). While achieving such features is not possible in current technologies, their implementation would open new possibilities not only for verbally impaired users, but rather the public at large.

### SUMMARY

In a first embodiment, a smart glass includes a frame, two eyepieces mounted on the frame, at least one microphone and a speaker, mounted on the frame, a camera, a memory configured to store multiple instructions, and a processor configured to execute the instructions, wherein the instructions comprise to provide to a user an environmental context from a signal provided by the microphone and the camera.

In a second embodiment, a computer-implemented method includes collecting, from a headset or wearable device with a user, a sensor signal indicative of a user environment, identifying the user environment based on a signal attribute, and communicating, to the user, a context for the user environment, in the headset.

In a third embodiment, a non-transitory, computer-readable medium stores instructions which, when executed by a processor, cause a computer to perform a method. The method includes collecting, from a headset or wearable device with a user, a sensor signal indicative of a user environment, identifying the user environment based on a signal attribute, and communicating, to the user, a context for the user environment, in the headset.

In yet other embodiments, a system includes a first means to store instructions, and a second means to execute the instructions and cause the system to perform a method, the method includes collecting, from a headset or wearable device with a user, a sensor signal indicative of a user environment, identifying the user environment based on a signal attribute, and communicating, to the user, a context for the user environment, in the headset.

These and other embodiments will be recognized by one of ordinary skill in the art in light of the following.

### BRIEF DESCRIPTION OF THE FIGURES

FIG. 1 illustrates an architecture including one or more wearable devices coupled to one another, to a mobile device, a remote server and to a database, according to some embodiments.

FIG. 2 illustrates a selection of a direction of arrival of an audio source from multiple microphones on a smart glass, according to some embodiments.

FIG. 3 illustrates a block diagram for providing auditory environmental context to impaired users, according to some embodiments.

FIG. 4 illustrates a block diagram to provide visual environmental context to impaired users, according to some embodiments.

FIG. 5 illustrates a block diagram for pairing speech to text capabilities to user hearing, according to some embodiments.

FIG. 6 illustrates a block diagram for providing customizable audio to impaired users, according to some embodiments.

FIG. 7 is a flowchart illustrating steps in a method for incorporating speech recognition in an immersive reality environment, according to some embodiments.

FIG. 8 is a block diagram illustrating an exemplary computer system with which headsets and other client devices, and the methods in FIG. 7, can be implemented.

In the figures, elements having the same or similar label number have features and attributes related to the same or similar attributes, unless explicitly stated otherwise.

### DETAILED DESCRIPTION

In the following detailed description, numerous specific details are set forth to provide a full understanding of the present disclosure. It will be apparent, however, to one ordinarily skilled in the art, that embodiments of the present disclosure may be practiced without some of these specific details. In other instances, well-known structures and techniques have not been shown in detail so as not to obscure the disclosure.

Users having speech and hearing disabilities are typically left out of the market of electronic appliances such as networked wearable devices for immersive reality applications. This is mostly due to the challenges involved in bringing these devices up to speed with the needs of such users, such as the ability to understand and comprehend the full context of an immersive reality situation (e.g., surrounding noises and background, environment, and the like).

Embodiments as disclosed herein provide technical solutions to the above technical problem arising in the realm of networked wearable devices for immersive reality applications. To do this, some embodiments use multiple sensors mounted on a headset or smart glass to capture background and/or environmental inputs. In addition, some embodiments take the advantage of fast networking strategies with paired mobile devices and networked servers to provide the sensor inputs to artificial intelligence (AI) servers that are trained to provide calibrated responses to different stimuli, for the users.

In 2011, the World Health Organization (WHO) estimated 1 billion (approx. 1 in 7) people live with a disability. More than 50 million Americans have a disability. The disability community consists of five disability cohorts: Deaf and Hard of Hearing, Speech Impairments and Loss, Cognitive and Learning, Mobility, and Vision Impairment. People with disabilities face higher barriers to health and fitness than the general population. In addition, these people face enormous physical, social, and attitudinal barriers toward their participation in physical and recreational activities. Disabilities can be permanent (e.g., congenital, accidental, veteran injuries, and the like), temporary (e.g., a broken arm), or situational (e.g., juggling groceries or carrying a child). In addition to the 1 billion people experiencing a permanent disability, designing appliances with disabled-person accessibility in mind, e.g., “Design for Inclusion,”—DFI—ensures product reach by diverse customer groups, and in a variety of situations.

Embodiments as disclosed herein are directed to sensory translation with wearable devices such as a smart glass or headset, or a wristband device, to provide experience equity and resolve the above problem. Some embodiments incorporate exercise/health experiences previously delivered visually (e.g., screens displaying heartrate/breathing/pedometer/etc.) into audio for smart glasses used by a sight impaired audience. Accordingly, embodiments as disclosed herein fill an important gap to level the field between people with disabilities and the rest of society.

Some embodiments may include third-party services that provide real-time human support on navigation, shopping, standing in line and following, and many daily tasks for blind people. Some embodiments include the ability to train a speech recognition model to a unique speech pattern of a user or an onlooker. Upon identification of an onlooker, the system may provide the onlooker identity to the user, to facilitate one-to-one communication. Other embodiments include alternatives to long-format verbal interactions, e.g., instead of starting everything with ‘Hey You,’ having a push to talk-to-Assistant mode, or even completely wake word-less mode, and ensuring voice access has alternative input modalities. Some embodiments include real-time closed captioning and keyboard input to help those who are deaf or hard of hearing.

DFI embodiments as disclosed herein bridge application interface gaps on core flows, encompassing: device set up, device settings, use of hardware, audio, companion application, and the like. Hardware (HW) and software (SW) decisions are based on prioritizing application interface features. While smart glasses and VR/AR headsets may not replace medical devices (such as hearing aids), some embodiments aim to complement and enhance the product experience, productivity, and communication of users with disabilities. VR/AR devices and smart glasses are designed for intensive, all-day wearability, and are thus naturally configured to be accessible and avoid interfering with medical and assistive technologies a wearer might be using.

Some of the primary features in devices as disclosed herein include identifying and designing for cohorts where smart glasses can have the largest impact; maximizing model utility and usability for cohorts with largest interface feature gains, and mitigating interference with medical devices (hearing aids, cochlear implants, pacemakers, and the like). Other relevant desirable features include maximizing device utility and usability across all disability cohorts, and enhanced hearing capabilities for people with hearing loss through advanced audio features.

### Exemplary System Architecture

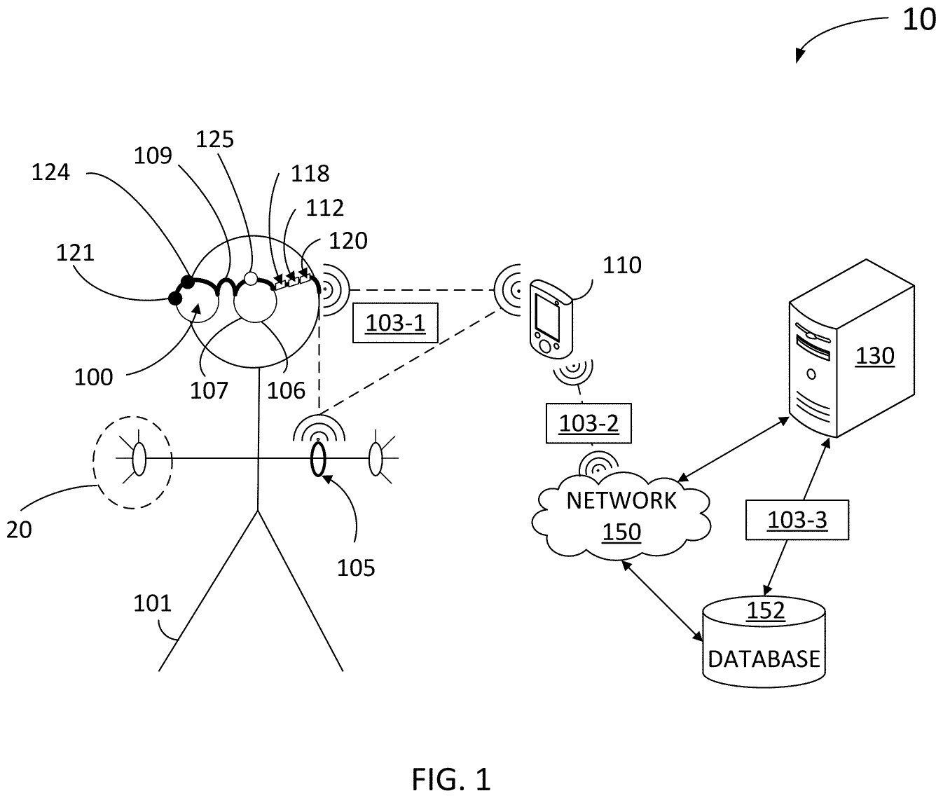

FIG. 1 illustrates an architecture 10 including one or more wearable devices (a smart glass 100 and a wristband device 105) coupled to one another, to a mobile device 110, a remote server 130 and to a database 152, according to some embodiments. Mobile device 110 may be a smart phone, all of which devices may communicate with one another via wireless communications and exchange a first dataset 103-1. Dataset 103-1 may include a recorded video, audio, or some other file or streaming media. The user 101 is also the owner or is associated with mobile device 110. User 101 makes a hand gesture 20, to communicate with an impaired interlocutor.

Mobile device 110 may be communicatively coupled with remote server 130 and database 152 via a network 150, and transmit/share information, files, and the like with one another (e.g., datasets 103-2 and 103-3).

In some embodiments, smart glass 100 may include a sensor 121 such as inertial measurement units (IMUs), gyroscopes, a microphone/speaker 124, cameras 125, and the like, mounted within a frame 109. Other sensors 121 that can be included in the wearable devices (e.g., smart glass 100, wrist-band 105, and the like) may be magnetometers, photodiodes, touch sensors, and other electromagnetic devices such as capacitive sensors, a pressure sensor, and the like. In some embodiments, smart glass 100 may include a display 107 on at least one eyepiece 106 to provide a model hand gesture to user 101, expressive of the speech from an interlocutor.

In addition, smart glass 100, or wrist-band 105, and any other wearable device, mobile device 110, server 130, and database 152 may include a memory circuit 120 storing instructions, and a processor circuit 112 configured to execute the instructions to cause smart glass 100 to perform, at least partially, some of the steps in methods consistent with the present disclosure. In some embodiments, memory 120 stores multiple hand gestures recognized for textual meaning for people with hearing disabilities.

In some embodiments, smart glass 100, wrist-band or wearable device 105, mobile device 110, server 130, and/or database 152 may further include a communications module 118 enabling the device to wirelessly communicate with remote server 130 via network 150. Smart glass 100 may thus download a multimedia online content (e.g., dataset 103-1) from remote server 130, to perform at least partially some of the operations in methods as disclosed herein. Network 150 may include, for example, any one or more of a local area network (LAN), a wide area network (WAN), the Internet, and the like. Further, the network can include, but is not limited to, any one or more of the following network topologies, including a bus network, a star network, a ring network, a mesh network, a star-bus network, tree or hierarchical network, and the like.

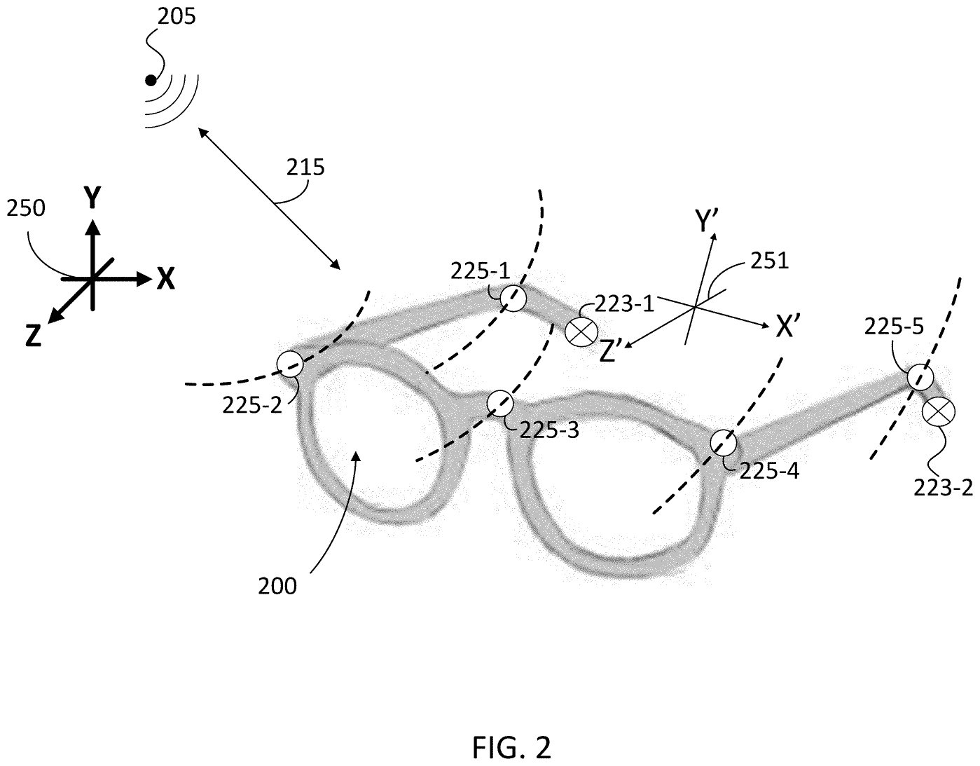

FIG. 2 illustrates a selection of a direction of arrival (DA) 215 of an audio source 205 from multiple microphones 225-1, 225-2, 225-3, 225-4, and 225-5 (hereinafter, collectively referred to as microphone array 225, e.g., mic 225-1, mic 225-2, mic 225-3, mic 225-4, and mic 225-5) on smart glass 200, according to some embodiments. Accordingly, DA 215 may be selected based on the difference in time of arrival of a sound waveform to each of the spatially distributed microphone array 225 on smart glass 200. In some embodiments, it may suffice to know the difference in time of arrival to assess DA 215 as a unit vector having two direction cosines. In some embodiments, the system may be able to determine a specific location of acoustic source 205 relative to smart glass 200 and even relative to geocoordinates. Smart glass 200 may also include speakers 223-1 and 223-2 (hereinafter, collectively referred to as “speakers 223”), configured to produce a stereo sound from audio source 205 along DA 215. In some embodiments, DA 215 is a vector oriented relative to either one of a world frame 250 and a glass frame 251, which frames may be oriented arbitrarily relative to each other.

In some embodiments, the assessment of DA 215 and location of acoustic source 205 may include resolving a linear regression problem associating times of arrival or sound signals to each of microphone array 225 based on the DA 215 and a speed of sound. To determine the time of arrival, the system may be configured to select a characteristic portion of the waveform generated by acoustic source 205, that may be easily identifiable using digital filters at each of microphone array 225. In some embodiments, and to enhance accuracy, the entire waveform or a substantive portion of it may be used to match the acoustic source origin. Other filtering techniques using hardware or software may be implemented, to identify distinct acoustic sources involved in any given event. In some embodiments, the software may include non-linear techniques such as non-linear regression, neural networks, machine learning, and artificial intelligence. Accordingly, in some embodiments, the system may include geolocation sensors and devices (e.g., IMU sensor 121) to better identify location and distances in the environment at a time of the event recording. The glass frame and the world frame are illustrated, showing a slight relative shift between the two due to a smart glass movement.

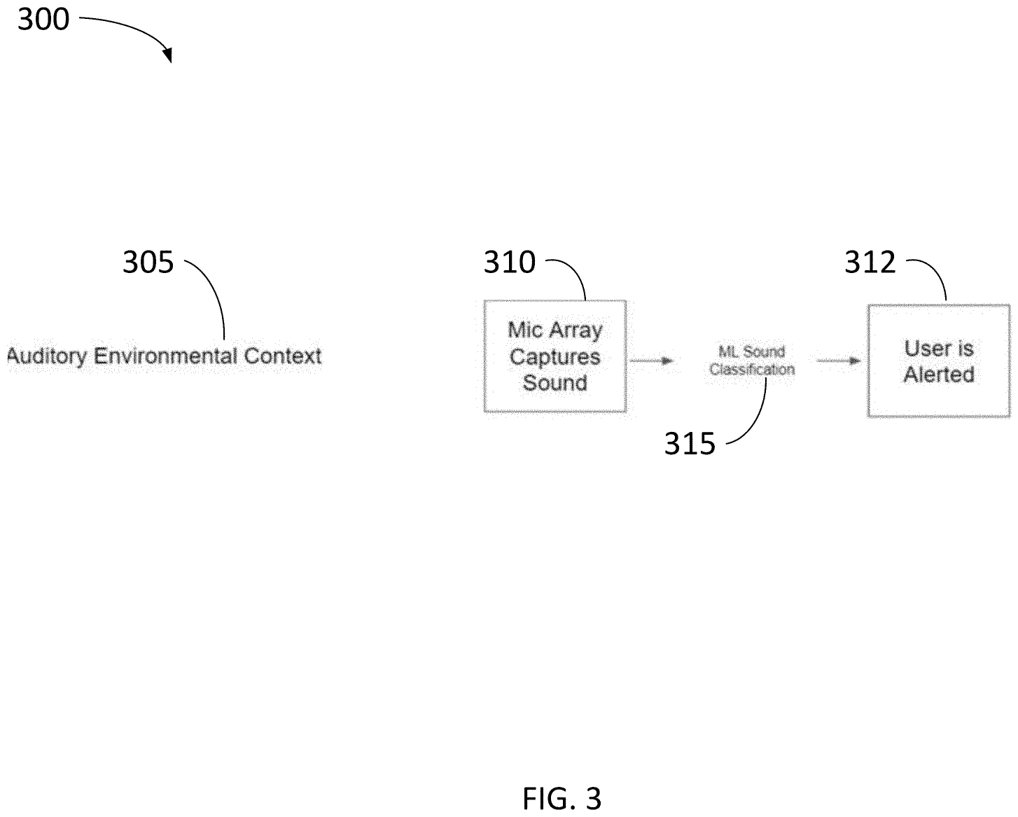

FIG. 3 illustrates a block diagram 300 for providing auditory environmental context 305 to impaired users, according to some embodiments. In block 310, a microphone array captures sound. An ML algorithm 315 performs sound classification, and in block 312, the user is alerted (e.g., the sound is a car approaching, or some other environmental hazard). In some embodiments, ML 315 identifies the voice of a known person, and block 312 includes informing the user about the person's identity.

In some embodiments, block diagram 300 works as an “Audio Guardian” for the user by giving the user spatial audio cues (e.g., “Knock at door”). The target cohort for such embodiments may include deaf and hard of hearing people.

In some embodiments, the system can be set to auto-detect specific environmental sounds such as smoke alarms, fire alarms, crying, etc. In some embodiments, the system may detect ambient sound/noise levels, direction, and volume, in addition to a sound classifier, and advise the user so that she/he can adjust speech levels, behavior, and other interactions to the environmental conditions. In some embodiments, a microphone mounted on the frame of the smart glass can be used to pick up environmental sounds and devices (portal calls) and phone rings/notifications. This is especially useful for sounds that users identify as “important” or “emergency” sounds like alarms, gunshots, baby crying, etc. Different users may have different kinds of hearing disabilities: some users may be able to hear out of one ear, not both, some users may not hear low noises, or some users may be affected by high volume (with a varying degree of severity). Accordingly, some embodiments may take into account these differences via automated ML algorithms, or by user-adjustable settings.

Some embodiments may include music detection (e.g., to determine whether there is ambient music playing that suggests an intended mood for the environment). This environmental awareness is highly desirable for people who are deaf. Additionally, in some embodiments, ML algorithm 315 may include AI-based crowd noise detection. Accordingly, the user may be alerted when a large group expresses something in unison (a threat, cheer, or a boo). This feature may improve the safety or sense of inclusion for hearing impaired users. Other types of alerts in block 312 may include an ambient volume level notification (e.g., user self-awareness: “am I in a loud or quiet place?”) or “is the sound getting louder (coming closer) or softer (moving away)?”

Some advantages of the system in block diagram 300 include the ability for a deaf person to set the volume on audio devices in a room for their kids or family or friends. The user can be informed via haptic feedback via wearables or phone pairing. In some embodiments, this feature is user-selectable: ring/vibe, app notification, or phone LED indication (e.g., using a flashlight strobe effect). For example, a “wearable” (wristband/watch) solution would actually make more sense but a paired phone would be more universal.

In some embodiments, block diagram 300 may include an option to indicate sound directionality to answer questions such as “Who's talking to me?” “where did that loud sound come from?”, “is the sound location moving relative to my position (like an ambulance crossing in front of me with siren on)?”, and the like.

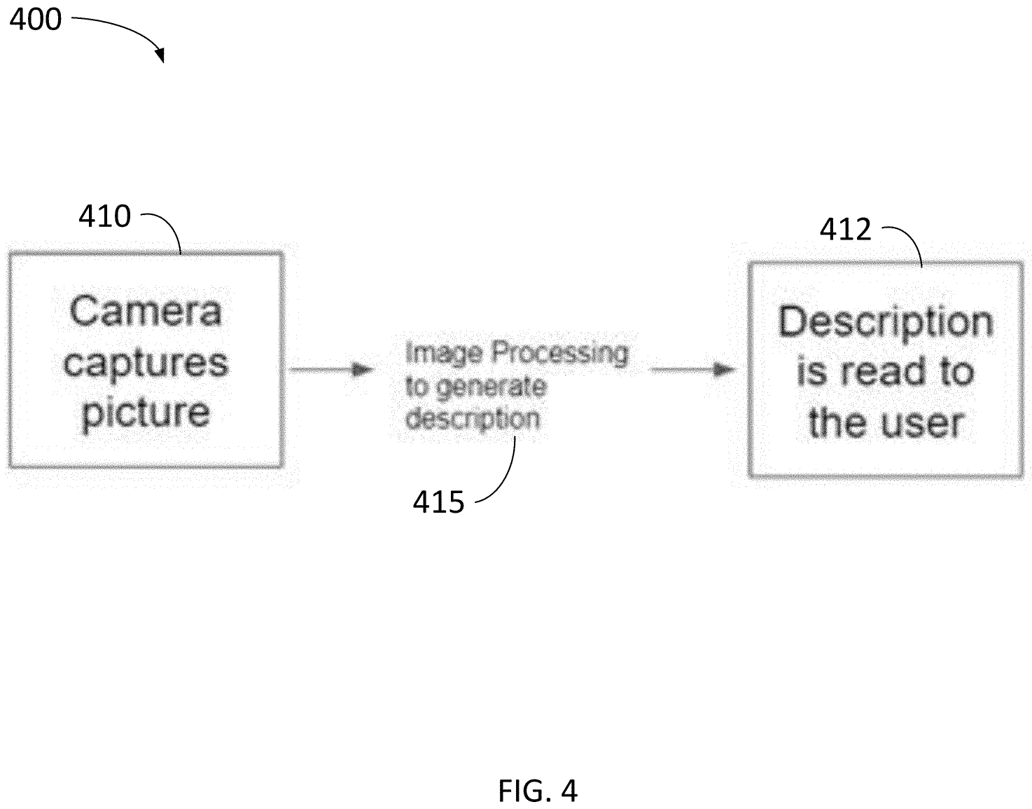

FIG. 4 illustrates a block diagram 400 to provide visual environmental context to impaired users, according to some embodiments. In block 410, a camera captures a picture of the user environment. Image processing software 415 generates a description (e.g., a textual caption) based on salient attributes of the picture. Image processing software 415 uses object recognition technology to generate a description of photos so the user hears a list of items contained in the picture as the scene is displayed on the smart glass. In block 412, the description is read to the user (e.g., via a speaker in the smart glass). In some embodiments, the description is provided as a text on the display in one of the eyepieces of the smart glass, when the user is able to read. A target cohort of a system in block diagram 400 may include blind/low vision/low mobility people. It is expected that ˜8% of wearable device users may have difficulty seeing even if wearing glasses, and ˜6.4% users may have mobility difficulties. Accordingly, block diagram 400 provides such users the experience of being with someone that can describe a scene.

In some embodiments, image processing software 415 is in a remote server, and so the smart glass provides the picture to the remote server and then receives the description 412 from the remote server via a network communication.

In some embodiments, a camera in the smart glass may be configured to extract different parts of the recorded video that it detects as significant/shareable. The camera may be powered by AI photo/video capture software, avoiding the need of camera pointing precision. In some embodiments, block diagram 400 provides real-time indications/narratives to a vision impaired wearer (e.g., people), and with appropriate permissions, even an assistant announcing the name of the person approaching (e.g., ‘name hints’), hazards, and the like.

In some embodiments, block 410 may include capturing content such as recording a video panning the room or taking a wide angle lens photo (helpful for people with limited mobility). In some embodiments, AI processing may identify people, objects, and events and extract them as “Moments” including: Photos of people and other subjects (with appropriate permissions and privacy settings considerations), photos of objects or noteworthy scenery, or videos of events like a baby dancing or laughing. In some embodiments, block 410 may include saving/sharing content such that users can save the generated photos/videos to share with others.

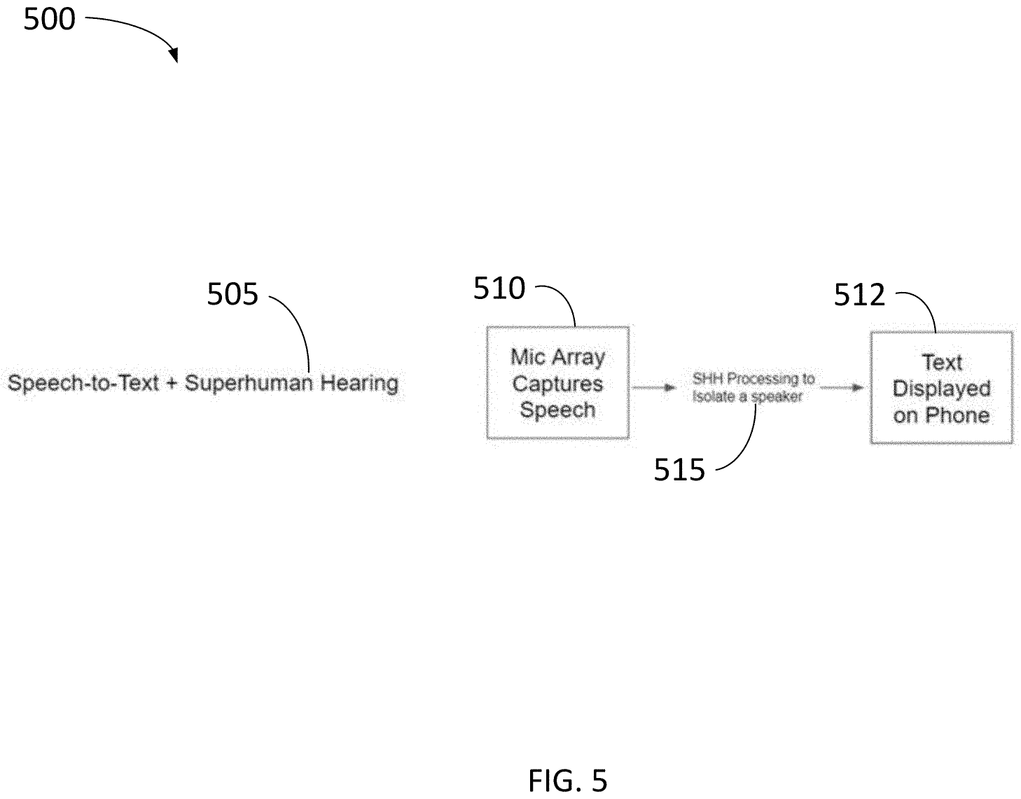

FIG. 5 illustrates a block diagram 500 for pairing speech to text (STT) capabilities 505 to user hearing, according to some embodiments. In block 510, a microphone array captures speech. A “super-human hearing” (SHH) algorithm 515 isolates a selected voice based on spectral signatures of a waveform. In block 512, the speech from the selected voice is converted into text and displayed for the user (e.g., in the screen of a mobile device with the user, or in a display on one of the eyepieces of the smart glass). In some embodiments, the text from the isolated voice may be read to the user via a speaker (e.g., when the user is unable to read from a display). The target cohort for activating block diagram 500 may include deaf and hard of hearing people. Additionally activating block diagram 500 may be useful for those with vision, physical, or cognitive impairments.

In some embodiments, SHH 515 includes speech recognition (ASR) applications running in a mobile device paired to the smart glass. The vocabulary may be optimized for commands and messaging, or adjusted for general speech, depending on the context. The microphones in the smart glass may be highly optimized for a speaker's voice. In some embodiments, block diagram 500 may be activated as a downstream feature of conversational focus. Block 512 could pipe conversationally-focused audio (e.g., via beam forming with microphone array 225) directly to an ASR on the mobile phone.

Combining speech isolation (enhanced hearing) with SHH 515 also cleans up the speech signal for a more accurate conversion of speech to text in block 512. Pairing enhanced hearing with STT 505 allows users to capture far-field speech and to distinguish STT 505 from different directions and/or speakers. Some embodiments pipe this feature into a language translation engine. Additionally, the translated text could be converted back to speech, in real time.

In some scenarios for activating block diagram 500, a deaf person wearing the smart glasses approaches a person speaking, to translate their voice into text. Even in a noisy environment, the user may naturally remain close to the person speaking, avoiding socially uncomfortable or unacceptable situations. Other configurations for activating block diagram 500 may include speaker identification (whether through voiceprint, wearer voice activity detection, direction of arrival, camera-based talker ID, and the like). This is useful when more than one person is speaking, including potentially the glasses wearer—to avoid self-transcription.

In some embodiments, the smart glass may include a high-end microphone for speech-to-text at a further distance, displaying text on the user's phone or even on the smart glass display. High-end techniques may include beamforming (for better speech pick up, cf. microphone array 225) and applying spatial captioning/labeling of multiple speakers in a scene or multi-party conversation, through the smart glasses.

In some embodiments, block diagram 500 is used to convert an AR voice to text. Some embodiments may combine the conversational focus with the audio superpowers from SHH software 515. This may be the case when the environment is so loud that the smart glasses can't safely deliver amplified content: instead, switch to STT 505.

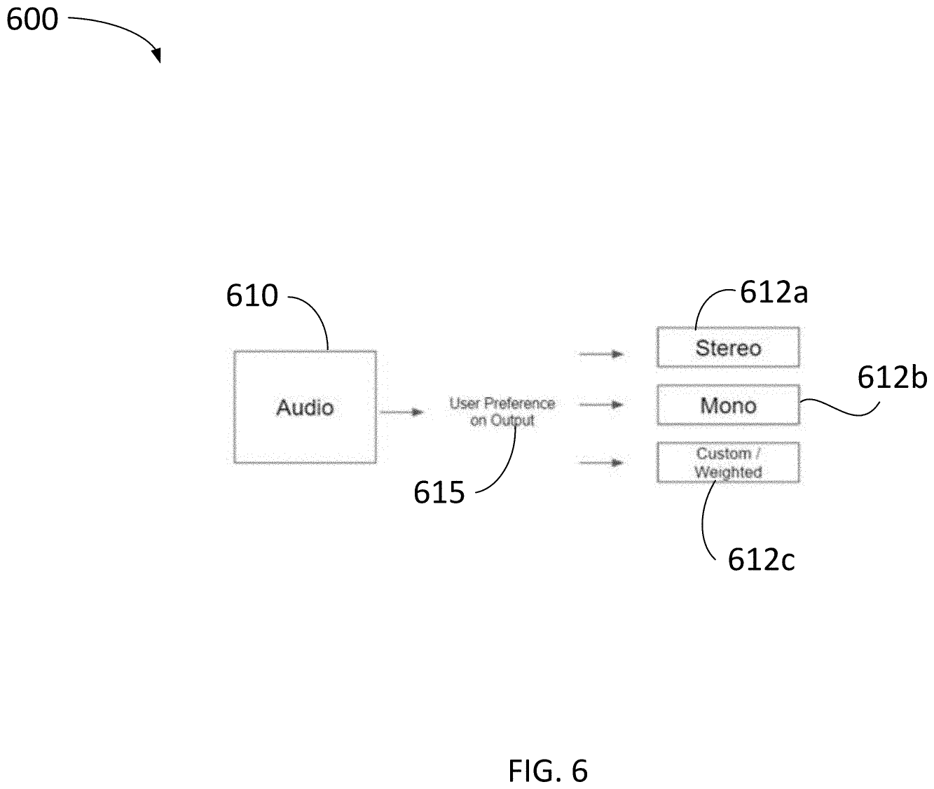

FIG. 6 illustrates a block diagram 600 for providing customizable audio to impaired users, according to some embodiments. Block 610 provides audio to the user, who has selected user preferences on output in block 615. Accordingly, the output may include any one of a stereo output 612a, a mono output 612b (e.g., when the user only hears from one ear), or a custom/balanced output 612c (e.g., when the user has partial hearing loss in one ear and desires a higher volume through the associated channel, hereinafter, collectively referred to as “audio outputs 612”). The target cohort for activating block diagram 600 includes deaf and hard of hearing people and users that have a loss of hearing on one ear who prefer mono-audio output. Activation of block diagram 600 provides more flexibility in ear orientation for those with asymmetrical hearing loss.

For some people, spatial audio is distracting so it may be desirable to enable users to narrow the sound field. In some embodiments, a user may prefer to focus on a given audio signal and not be distracted with stereo sound. For example, instead of hearing a stereo sound, a user may prefer to hear an announcement “sound from 5 o'clock.” Also, activation of block diagram 600 provides users control over whether the sound field changes based on their head orientation, or stay fixed no matter how they turn their head.

In some embodiments, user preferences 615 may include sound funneling capabilities with presets for optimizing voice frequencies, or for lowering ambient noise for people who are noise sensitive. In some embodiments, a device option may include stereo, L/R with adjustable weights (for asymmetric hearing loss), and mono, and the ability to funnel spatial audio representative of L/R fields into customizable outputs. For example, people with hearing loss out of one ear will tend to position themselves strategically to best capture sound/conversations (e.g., sitting at a corner, tilting/rotating the head, etc.). Sound funneling from activating block diagram 600 provides the user more orientation freedom while still capturing the intended sound.

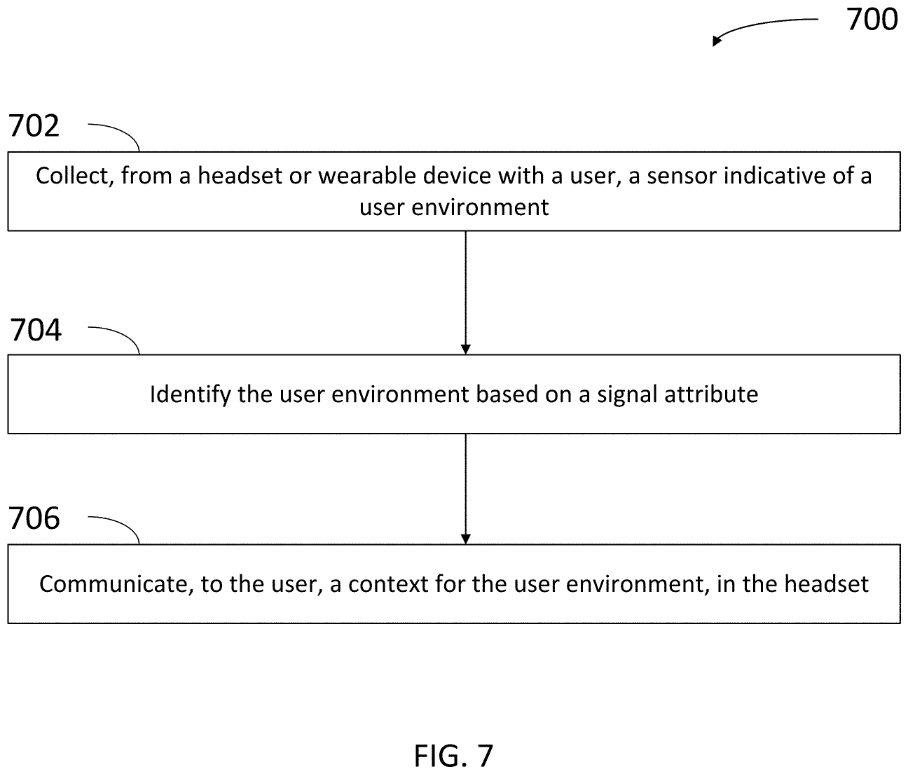

FIG. 7 is a flowchart illustrating steps in a method 700 for incorporating speech recognition in an immersive reality environment, according to some embodiments. In some embodiments, at least one or more of the steps in method 700 may be performed by a processor executing instructions stored in a memory in either one of a smart glass or other wearable device on a user's body part (e.g., head, arm, wrist, leg, ankle, finger, toe, knee, shoulder, chest, back, and the like). In some embodiments, at least one or more of the steps in method 700 may be performed by a processor executing instructions stored in a memory, wherein either the processor or the memory, or both, are part of a mobile device for the user, a remote server or a database, communicatively coupled with each other via a network. Moreover, the mobile device, the smart glass, and the wearable devices may be communicatively coupled with each other via a wireless communication system and protocol (e.g., radio, Wi-Fi, Bluetooth, near-field communication—NFC—and the like). In some embodiments, a method consistent with the present disclosure may include one or more steps from method 700 performed in any order, simultaneously, quasi-simultaneously, or overlapping in time.

Step 702 includes collecting, from a headset or wearable device with a user, a sensor signal indicative of a user environment. In some embodiments, step 702 includes collecting an image from a camera mounted on the headset and identifying the user environment comprises determining a textual description of the image. In some embodiments, step 702 includes collecting an image from a camera mounted on the headset, and communicating a context for the user environment comprises providing, via a speaker, a spoken description of the image from the camera. In some embodiments, step 702 includes collecting a background sound with a microphone, and communicating a context for the user environment comprises removing the background sound from a sound signal provided to the user via a speaker. In some embodiments, step 702 includes collecting multiple audio signals from a microphone array, identifying a direction of a selected sound source by synchronizing a time delay between the audio signals for a waveform associated with the selected sound source, and enhancing the audio signal from the selected sound source.

Step 704 includes identifying the user environment based on a signal attribute. In some embodiments, the sensor signal is a human voice from a microphone, and step 704 includes identifying the human voice from the microphone.

Step 706 includes communicating, to the user, a context for the user environment, in the headset. In some embodiments, the sensor signal is a broadband spectral sound from a microphone, the signal attribute is a spectral profile of the broadband spectral sound, and step 706 includes a context for the user environment and comprises converting the spectral profile into a narrow band spectral sound that can be heard by the user. In some embodiments, step 706 includes providing the user a name of a person associated with the human voice. In some embodiments, the sensor signal is a sound waveform including multiple voices for multiple people, the signal attribute is a voice for each person, and step 706 includes adding a caption with a name for each person in a headset display. In some embodiments, the sensor signal is a sound waveform including multiple people's voices, and step 706 includes displaying a transcript of at least one of the people's voices on a headset display. In some embodiments, the sensor signal is a sound waveform including a speech in a language that is foreign to the user, and step 706 includes translating the speech into a language selected by the user.

### Hardware Overview

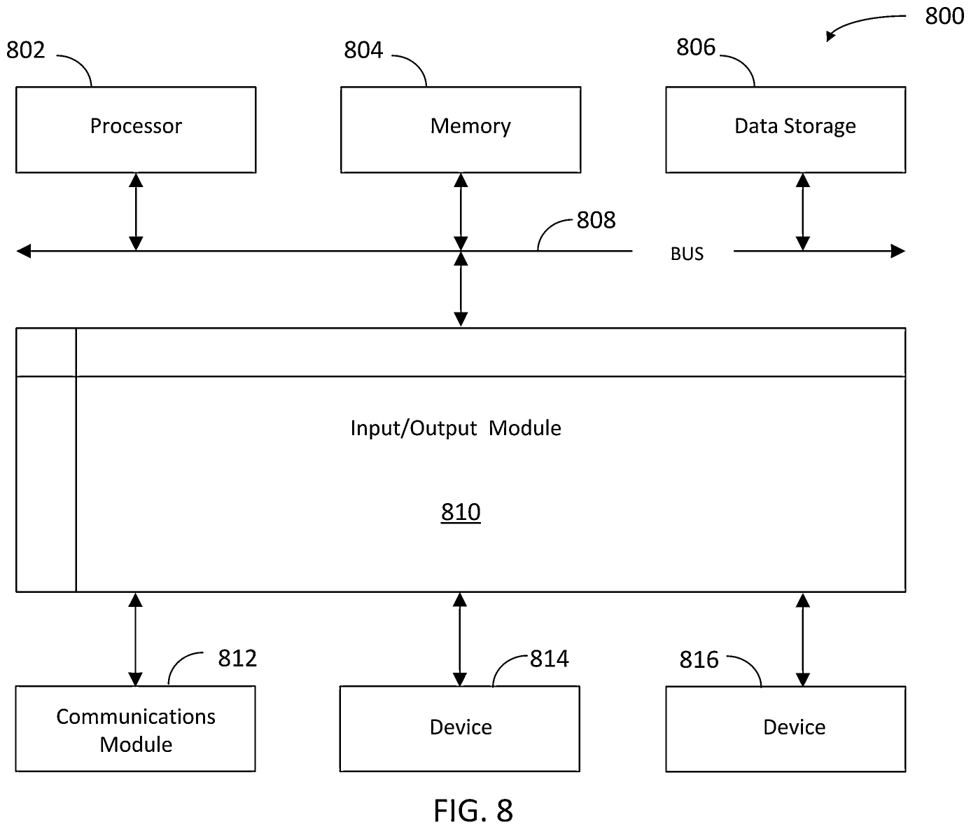

FIG. 8 is a block diagram illustrating an exemplary computer system 800 with which headsets and other client devices 110, and method 700 can be implemented, according to some embodiments. In certain aspects, computer system 800 may be implemented using hardware or a combination of software and hardware, either in a dedicated server, or integrated into another entity, or distributed across multiple entities. Computer system 800 may include a desktop computer, a laptop computer, a tablet, a phablet, a smartphone, a feature phone, a server computer, or otherwise. A server computer may be located remotely in a data center or be stored locally.

Computer system 800 includes a bus 808 or other communication mechanism for communicating information, and a processor 802 (e.g., processor 112) coupled with bus 808 for processing information. By way of example, the computer system 800 may be implemented with one or more processors 802. Processor 802 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 800 can include, in addition to hardware, code that creates an execution environment for the computer program in question, e.g., code that constitutes processor firmware, a protocol stack, a database management system, an operating system, or a combination of one or more of them stored in an included memory 804 (e.g., memory 120), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled with bus 808 for storing information and instructions to be executed by processor 802. The processor 802 and the memory 804 can be supplemented by, or incorporated in, special purpose logic circuitry.

The instructions may be stored in the memory 804 and implemented in one or more computer program products, e.g., one or more modules of computer program instructions encoded on a computer-readable medium for execution by, or to control the operation of, the computer system 800, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, metaparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, with languages, and xml-based languages. Memory 804 may also be used for storing temporary variable or other intermediate information during execution of instructions to be executed by processor 802.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 800 further includes a data storage device 806 such as a magnetic disk or optical disk, coupled with bus 808 for storing information and instructions. Computer system 800 may be coupled via input/output module 810 to various devices. Input/output module 810 can be any input/output module. Exemplary input/output modules 810 include data ports such as USB ports. The input/output module 810 is configured to connect to a communications module 812. Exemplary communications modules 812 include networking interface cards, such as Ethernet cards and modems. In certain aspects, input/output module 810 is configured to connect to a plurality of devices, such as an input device 814 and/or an output device 816. Exemplary input devices 814 include a keyboard and a pointing device, e.g., a mouse or a trackball, by which a consumer can provide input to the computer system 800. Other kinds of input devices 814 can be used to provide for interaction with a consumer as well, such as a tactile input device, visual input device, audio input device, or brain-computer interface device. For example, feedback provided to the consumer can be any form of sensory feedback, e.g., visual feedback, auditory feedback, or tactile feedback; and input from the consumer can be received in any form, including acoustic, speech, tactile, or brain wave input. Exemplary output devices 816 include display devices, such as an LCD (liquid crystal display) monitor, for displaying information to the consumer.

According to one aspect of the present disclosure, headsets and client devices 110 can be implemented, at least partially, using a computer system 800 in response to processor 802 executing one or more sequences of one or more instructions contained in memory 804. Such instructions may be read into memory 804 from another machine-readable medium, such as data storage device 806. Execution of the sequences of instructions contained in main memory 804 causes processor 802 to perform the process steps described herein. One or more processors in a multi-processing arrangement may also be employed to execute the sequences of instructions contained in memory 804. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., a data server, or that includes a middleware component, e.g., an application server, or that includes a front end component, e.g., a client computer having a graphical consumer interface or a Web browser through which a consumer can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 800 can include clients and servers. A client and server are generally remote from each other and typically interact through a communication network. The relationship of client and server arises by virtue of computer programs running on the respective computers and having a client-server relationship to each other. Computer system 800 can be, for example, and without limitation, a desktop computer, laptop computer, or tablet computer. Computer system 800 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 802 for execution. Such a medium may take many forms, including, but not limited to, non-volatile media, volatile media, and transmission media. Non-volatile media include, for example, optical or magnetic disks, such as data storage device 806. Volatile media include dynamic memory, such as memory 804. Transmission media include coaxial cables, copper wire, and fiber optics, including the wires forming bus 808. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

To illustrate the interchangeability of hardware and software, items such as the various illustrative blocks, modules, components, methods, operations, instructions, and algorithms have been described generally in terms of their functionality. Whether such functionality is implemented as hardware, software, or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public, regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A smart glass, comprising:
a frame;
two eyepieces mounted on the frame;
at least one microphone and a speaker, mounted on the frame;
a camera;
a memory configured to store multiple instructions; and
a processor configured to execute the instructions, wherein the instructions comprise to provide to a user an environmental context from a signal provided by the microphone and the camera.

2. The smart glass of claim 1, further comprising a communications module configured to communicate with a wearable device of the user, wherein the wearable device provides an environmental data to the processor.

3. The smart glass of claim 1, further comprising a communications module configured to communicate the signal provided by the microphone and the camera to a mobile device, and the mobile device displays the environmental context on a screen, for the user.

4. The smart glass of claim 1, further comprising a communications module configured to communicate the signal provided by the microphone and the camera to a network server, and to receive from the network server the environmental context.

5. The smart glass of claim 1, wherein at least one of the eyepieces includes a display configured to provide the environmental context to the user as a readable text.

6. The smart glass of claim 1, wherein the speaker is configured to provide the environmental context to the user as an audio description.

7. The smart glass of claim 1, wherein the microphone includes an array configured to capture a stereo sound and the processor provides an alert to the user about a direction of a sound source based on the stereo sound.

8. The smart glass of claim 1, wherein the microphone includes an array configured to capture a stereo sound and the processor converts the stereo sound to a mono-audio output from the speaker for a user that has diminished hearing in one ear.

9. The smart glass of claim 1, wherein the microphone includes an array configured to capture a stereo sound and the processor identifies a direction of a source associated with a waveform in the stereo sound, and at least one of the eyepieces includes a display that labels the source associated with the waveform.

10. The smart glass of claim 1, wherein the camera is configured to collect a picture of the environmental context, the processor executes instructions in the memory to obtain a textual description of the picture, and to cause the speaker to read the textual description of the picture to the user.

11. A computer-implemented method, comprising:
collecting, from a headset or wearable device with a user, a sensor signal indicative of a user environment;
identifying the user environment based on a signal attribute; and
communicating, to the user, a context for the user environment, in the headset.

12. The computer-implemented method of claim 11, wherein collecting a sensor signal comprises collecting an image from a camera mounted on the headset, and identifying the user environment comprises determining a textual description of the image.

13. The computer-implemented method of claim 11, wherein collecting a sensor signal comprises collecting an image from a camera mounted on the headset, and communicating a context for the user environment comprises providing, via a speaker, a spoken description of the image from the camera.

14. The computer-implemented method of claim 11, wherein collecting a sensor signal comprises collecting a background sound with a microphone, and communicating a context for the user environment comprises removing the background sound from a sound signal provided to the user via a speaker.

15. The computer-implemented method of claim 11, wherein collecting the sensor signal comprises collecting multiple audio signals from a microphone array, identifying a direction of a selected sound source by synchronizing a time delay between the audio signals for a waveform associated with the selected sound source, and enhancing the audio signal from the selected sound source.

16. The computer-implemented method of claim 11, wherein the sensor signal is a broadband spectral sound from a microphone, the signal attribute is a spectral profile of the broadband spectral sound, and communicating a context for the user environment comprises converting the spectral profile into a narrow band spectral sound that can be heard by the user.

17. The computer-implemented method of claim 11, wherein the sensor signal is a human voice from a microphone, wherein identifying the user environment based on the signal attribute comprises identifying the human voice from the microphone and communicating a context for the user environment comprises providing the user a name of a person associated with the human voice.

18. The computer-implemented method of claim 11, wherein the sensor signal is a sound waveform including multiple voices for multiple persons, the signal attribute is a voice for each person, and communicating a context for the user environment comprises adding a caption with a name for each person in a headset display.

19. The computer-implemented method of claim 11, wherein the sensor signal is a sound waveform including multiple people's voices, and communicating a context for the user environment comprises displaying a transcript of at least one of the people's voices on a headset display.

20. The computer-implemented method of claim 11, wherein the sensor signal is a sound waveform including a speech in a language that is foreign to the user, and communicating a context for the user environment comprises translating the speech into a language selected by the user.
